(19) United States
(12) Patent Application Publication
(10) Pub. No.: US 2026/0159814 A1
(43) Pub. Date: Jun. 11, 2026

(54) METHOD FOR PREPARING PORCINE INDUCED PLURIPOTENT STEM CELL

(71) Applicant: SIMPLE planet Co., Ltd., Seoul (KR)

(72) Inventors: Hee Ho Park, Seoul (KR); Ildoo Jeong, Seoul (KR); Dongoh Han, Seoul (KR); Ki Hyun Yoo, Seoul (KR); Kwan Hyun Kim, Seoul (KR); Bong Jong Seo, Seoul (KR); Jee Yoon Lim, Seoul (KR)

(73) Assignee: SIMPLE planet Co., Ltd., Seoul (KR)

(21) Appl. No.: 19/337,096

(22) Filed: Sep. 23, 2025

(30) Foreign Application Priority Data
Dec. 11, 2024 (KR) 10-2024-0184157
Apr. 21, 2025 (KR) 10-2025-0051444

(51) Int. Cl. C12N 5/074; C12N 5/077; C12N 13/00; C12N 15/87

(52) U.S. Cl. CPC C12N 5/0696; C12N 5/0658; C12N 13/00; C12N 15/87; C12N 2501/115; C12N 2506/02; C12N 2510/00

(57) ABSTRACT

The present invention relates to a method for preparing porcine induced pluripotent stem cells (piPSCs), wherein hOCT3, hOCT4, hSK, hUL, mP53DD, and EBNA1 genes are introduced into porcine embryonic fibroblasts by electroporation, thereby producing piPSCs without problems caused by the integration of foreign genes. The porcine induced pluripotent stem cells prepared by the preparation method of the present invention have pluripotency and spontaneous differentiation capability, and in particular, have excellent differentiation ability from piPSCs to myotube cells, and thus can be effectively used in the cultured meat industry.

### CROSS REFERENCE TO RELATED APPLICATIONS

This patent application claims the benefit of priority under 35 U.S.C. § 119 from Korean Patent Application No. 10-2024-0184157 filed Dec. 11, 2024, and Korean Patent Application No. 10-2025-0051444 filed Apr. 21, 2025, the entire contents of which are incorporated herein by reference.

### SEQUENCES

The nucleic and amino acid sequences are shown using standard letter abbreviations for nucleotide bases, and amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “7037-114089-01_Sequence.xml” (51,889 bytes), which was created on Sep. 16, 2025, which is incorporated by reference herein.

### BACKGROUND OF THE INVENTION

### 1. Field of the Invention

The present invention relates to a method for preparing porcine induced pluripotent stem cells, porcine induced pluripotent stem cells prepared by the method, and culture bodies differentiated therefrom.

### 2. Description of the Related Art

Induced pluripotent stem cells (iPSCs) are cells that have pluripotency, like embryonic stem cells, and have the ability to differentiate into multiple cell types. Induced pluripotent stem cells (iPSCs) are generated by introducing reprogramming factors into fully differentiated somatic cells to return them to a pluripotent state. Through this process, iPSCs can differentiate into various cell lineages, and these cells have the property of being able to proliferate almost indefinitely. In particular, the Yamanaka factors (Oct4, Sox2, c-Myc, and Klf4), which were first used by Professor Yamanaka's team to generate mouse iPSCs, play an important role in maintaining the pluripotency of iPSCs. Oct4 and Sox2 are key factors in maintaining pluripotency in the embryo, while Klf4 and c-Myc play important roles in maintaining the phenotype and proliferation of embryonic stem cells (ES cells). These factors are introduced into somatic cells via lentivirus or retrovirus, which has led to the development of mouse and human iPSCs.

Pigs are important as research subjects because they have similar physiological, behavioral, and psychological characteristics to humans. In particular, because pigs and humans show high genetic similarity, the generation of porcine induced pluripotent stem cells (piPSCs) is a very useful resource for medical research and life science development. Professor R. Michael Roberts' team successfully generated piPSCs by introducing Yamanaka factors into porcine somatic cells using lentivirus. Delivery methods using lentivirus and retrovirus are widely used due to their high efficiency and are commonly applied in the generation of piPSCs. However, virus-based methods often fail to form chimeras and have difficulties in removing reverse differentiation factors from somatic cells. This may act as a major factor that may cause problems in future clinical applications and therapeutic purposes.

As a prior art, a prior literature [Cell Cycle 17.23 (2018): 2547-2563] is a paper on a method for generating transgene-free porcine intermediate-type induced pluripotent stem cells, describing a non-integrative vector-based reverse differentiation method using porcine fetal fibroblasts. Specifically, the literature discloses a method for reverse-differentiating mammalian fibroblasts using episomal plasmids (hOCT3/4-shp53, hSOX2, hKLF4, hLIN28, and hL-MYC) through a single electroporation method. In addition, a prior literature [International Journal of Oral Biology 39.4 (2014)] is a paper on a method for generating patient-specific iPSC cell lines from peripheral blood mononuclear cells (PBMCs). It describes a method for isolating PBMCs, and generating iPSCs by introducing Sox2 and myc genes into the PBMCs through electroporation. In addition, a prior literature [International Journal of Stem Cells 16.1 (2023): 36-43] describes a method for generating induced pluripotent stem cells from lymphoblastoid cell lines (LCLs) using episomal vectors and electroporation. This literature describes a method to generate iPSCs by introducing hOCT3/4, hSK, hUL, and mp53DD genes into LCLs via electroporation using an oriP/EBNA-1-based episomal vector.

In cultured meat production, muscle cells are an important component. Research has been actively conducted to differentiate porcine induced pluripotent stem cells (piPSCs) into various cell lineages, including hepatocytes, vascular endothelial cells, neural cells, and muscle cells, and in particular, differentiation into muscle cells plays an important role in cultured meat production. According to research, because muscle differentiation can be induced without chemical treatment, muscle cells are a major component of cultured meat. In previous studies, muscle differentiation was induced through lentivirus-mediated MyoD gene delivery, but studies on muscle differentiation by virus-free methods are limited. To date, muscle differentiation of piPSCs has all relied on virus-based methods. However, gene delivery via viruses carries the potential for unexpected consequences caused by the integrated genes, such as mutations, allergies, and toxins. These issues are raising concerns about the impact on human health.

As interest in animal welfare has increased in recent years, efforts to reduce the use of laboratory animals have also expanded. In addition, interest in cultured meat production has grown significantly in recent years due to increased meat consumption and environmental concerns. Cultured meat has the advantage of minimizing animal tests and enabling sustainable meat production. Muscle satellite cells are used to induce muscle differentiation in cultured meat production because they can induce muscle differentiation without chemical treatment. However, ensuring consistent cell uniformity in such technologies remains a challenge. In addition, the process of extracting cells from animals is subject to ethical and practical constraints, so new methods need to be developed.

Thus, the present inventors introduced hOCT3, hOCT4, hSK, hUL, mP53DD, and EBNA1 genes into porcine embryonic fibroblasts using electroporation, thereby generating piPSCs in which foreign genes are not integrated, and confirmed that piPSCs maintain pluripotency and exhibit spontaneous differentiation capacity even in the absence of pluripotency genes. In addition, the present inventors induced muscle differentiation in piPSCs and confirmed that piPSCs differentiate stepwise into myogenic progenitors, myoblasts, and myotubes, thus completing the present invention.

### SUMMARY OF THE INVENTION

It is an object of the present invention to provide a method for preparing porcine induced pluripotent stem cells (piPSCs).

It is another object of the present invention to provide porcine induced pluripotent stem cells prepared by the method of the present invention.

It is another object of the present invention to provide a cell composition comprising at least one of embryoid bodies, myogenic progenitors, myoblasts, or myotube cells differentiated from the porcine induced pluripotent stem cells obtained by the preparation method of the present invention.

To achieve the above objects, the present invention provides a method for preparing porcine induced pluripotent stem cells (piPSCs) comprising the following steps:
1) a step of transforming porcine embryonic fibroblasts (PEF) with an episomal vector by electroporation to obtain transformed cells, wherein, the episomal vector is a vector expressing one or more genes selected from the group consisting of hOCT3/4, hSK, hUL, mP53DD, and EBNA1 genes; and
2) a step of reverse differentiating the obtained transformed cells and then forming piPSC cells or populations.

The present invention also provides porcine induced pluripotent stem cells prepared by the method described above.

In addition, the present invention provides a cell composition comprising at least one of embryoid bodies, myogenic progenitors, myoblasts, or myotube cells differentiated from the porcine induced pluripotent stem cells obtained by the preparation method of the present invention.

### Advantageous Effect

The porcine induced pluripotent stem cells (piPSCs) prepared by the method according to the present invention can be stably maintained for at least 100 passages, thereby providing high stability and productivity, and since the episomal vector is not integrated into the cells, unexpected problems caused by foreign genes can be avoided, ensuring excellent safety. Due to these characteristics, the piPSCs have high potential for use in the cultured meat industry, and can also be useful as cell therapeutics based on their ability to differentiate into various cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram schematically illustrating the process of preparing piPSCs using electroporation according to the present invention.

FIG. 2 is a diagram briefly illustrating the process of preparing piPSCs.

FIG. 3 is a diagram showing the morphology of piPSCs cultured by a chemical separation method.

FIG. 4 is a diagram showing the morphology of piPSCs cultured by a mechanical passage method.

FIG. 5A and FIG. 5B are showing the results of AP staining of piPSCs present in the STO feeder layer as visual images and a quantitative graph.

FIG. 6 is a diagram showing the microscope images of piPSC colonies stained with AP.

FIG. 7 is a diagram showing the RT-PCR results indicating that the episomal vector has not been integrated into the cell genome.

FIGS. 8A and 8B are showing the RT-PCR results for pluripotency markers (Oct4, Sox2, Klf4, and c-Myc).

FIG. 9 is a diagram showing the protein expression levels of the pluripotency markers OCT4 and NANOG in piPSCs through immunocytochemistry (ICC).

FIG. 10 is a diagram showing the hanging drop method.

FIG. 11 is a diagram showing the spontaneous differentiation process.

FIG. 12 is a diagram showing the formation of porcine embryonic bodies (pEBs) from piPSCs by the hanging drop method.

FIG. 13 is a diagram showing the qRT-PCR results for genes related to the three germ layers in pEB.

FIG. 14 is a diagram showing the expression levels of TUJ1, α-SMA, and AFP proteins related to the 3 germ layers through ICC.

FIG. 15 is a diagram showing the differentiation process from piPSCs to myotube cells.

FIG. 16 is a diagram showing the morphology of piPSCs and myogenic progenitors through microscope images.

FIG. 17 is a diagram showing the qRT-PCR results for the muscle progenitor cell markers Pax3 and Pax 7 genes.

FIG. 18A is a diagram showing the protein expression levels of PAX3 and PAX7, which are muscle progenitor cell markers, through ICC.

FIG. 18B is a graph showing the normalized results of the protein expression levels of PAX3 and PAX7, which are muscle progenitor cell markers, observed through ICC.

FIG. 19 is a diagram showing the differentiation morphology into myoblasts under three culture media conditions.

FIG. 20A is a diagram showing the protein expression levels of MYOD and MYF5, which are myoblast markers, through ICC.

FIG. 20B is a set of graphs showing the normalized results of the protein expression levels of MYOD and MYF5, which are myoblast markers, observed through ICC.

FIG. 21 is a diagram showing the morphological changes of cells during the differentiation of myogenic progenitors into myoblasts under the CM-M medium culture condition.

FIG. 22 is a diagram showing the results of qRT-PCR for marker genes of myogenic progenitors under the CM-M medium culture condition.

FIG. 23A is a diagram showing the protein expression levels of MYOD and MYF5, which are muscle progenitor cell markers, under the CM-M medium culture condition, through ICC.

FIG. 23B is a graph showing the normalized results of the protein expression levels of MYOD and MYF5, which are muscle progenitor cell markers, under the CM-M medium culture condition, observed through ICC.

FIG. 24 is a diagram showing the results of qRT-PCR for the myotube cell markers Myh1, Myh2, Myh7, Mrf4, and Desmin genes.

FIG. 25 is a diagram showing the expression level of MYH protein, which is abundantly present in muscle fibers, through ICC.

FIG. 26 is a diagram showing the expression of MYH protein in muscle fibers through ICC.

### DESCRIPTION OF THE PREFERRED EMBODIMENTS

Hereinafter, the present invention is described in detail.

The present invention provides a method for preparing porcine induced pluripotent stem cells (piPSCs) comprising the following steps:
1) a step of transforming porcine embryonic fibroblasts (PEF) with an episomal vector by electroporation to obtain transformed cells, wherein, the episomal vector is a vector expressing one or more genes selected from the group consisting of hOCT3/4, hSK, hUL, mP53DD, and EBNA1 genes; and
2) a step of reverse differentiating the obtained transformed cells and then forming piPSC cells or populations.

In the present invention, the episomal vector is an expression vector containing hOCT3, hOCT4, hUL, mP53DD, or EBNA1 gene.

In the present invention, the iPSC cells or populations obtained by transducing the six genes into porcine embryonic fibroblasts and then reverse differentiating them may exhibit high passage efficiency, however, if an episomal vector containing the mp53DD and EBNA1 genes is not introduced, the passage efficiency may be low.

Stem cells are cells that can continuously produce cells identical to themselves in an undifferentiated state and have the property of differentiating into various cells under appropriate conditions.

Stem cells can be broadly classified into embryonic stem cells and adult stem cells based on their differentiation capacity and time of production. Another classification can be divided into pluripotent, multipotent, and unipotent stem cells based on their differentiation capacity.

Pluripotent stem cells are stem cells that can differentiate into all three germ layers (endoderm, mesoderm, and ectoderm) that make up the body, meaning they can differentiate into any cell or organ tissue in the body. The term “stem cells” used in the present invention refers to cells having pluripotency or limited differentiation ability (multipotency) that can differentiate into cells closely related to a specific tissue or function.

The term “differentiation” used in the present invention means a phenomenon in which cells become specialized in structure or function while growing through division and proliferation, that is, a process in which non-specialized cells develop into specific cells, and particularly includes a process in which stem cells develop into specific cells.

The term “induced pluripotent stem cells (iPSCs)” (also referred to as “reverse differentiated stem cells”) used in the present invention means stem cells that are created by establishing undifferentiated stem cells with a differentiation ability similar to that of embryonic stem cells using a reverse differentiation technique on somatic cells of an animal, and have a differentiation ability similar to that of ESCs.

In the present invention, the porcine induced pluripotent stem cell line refers to a cell line of continuous passage, i.e., an established cell line, which means that cultured cells acquire infinite proliferative properties and become a cell line of continuous passage.

The method of the present invention may further comprises, after step 2, a step (step 3) of culturing the formed piPSC cells or populations with feeder cells to expand and maintain the piPSC cells or populations.

The formed piPSC cells or populations can be expanded and maintained by mechanical passage culture.

In the present invention, the term “subculture” means a method of continuously culturing cells in a healthy state for a long period of time by periodically transferring a portion of cells to a new culture vessel and then changing the culture medium to continuously culture the cells for generations. As the number of cells increases in a culture vessel with limited space, the cells will naturally die after a period of time due to the consumption of nutrients or the accumulation of contaminants, so subculture is used as a way to increase the number of healthy cells. At this time, replacing the medium (culture vessel) once or dividing the cell group and culturing it is usually called 1 passage.

In the present invention, the term “mechanical separation” means physically or mechanically separating a cell mass, and any method known in the art may be used without limitation as long as it is suitable for the purpose without using enzymatic treatment, but preferably, separation may be performed using a blade, a tissue chopper, a needle, a pipette, an embryoid body divider (EBD), or a scraper.

In the present invention, mechanical passaging can be performed every 3 to 15 days, preferably every 4 to 12 days, and more preferably every 5 to 10 days.

In the present invention, the electroporation of step 1) can be performed at a voltage condition of 600 V to 2500 V, preferably at a voltage condition of 1000 V to 2000 V, and more preferably at a voltage condition of 1300 V to 1800 V. In addition, the electroporation time may be from 1 ms to 20 ms, preferably from 4 ms to 16 ms, and more preferably from 8 ms to 12 ms. The electroporation may also be performed with 1 to 6 electric pulses, preferably 2 to 5 electric pulses. Outside the range of the above electroporation conditions, cytotoxicity may occur and transformation efficiency may be reduced.

In the present invention, the transformed cells in step 2) can be reverse-differentiated through a step of reverse differentiation comprising: I) a step of culturing in an initial culture medium of iPSCs for 1 to 15 days after electroporation; and II) a step of culturing in an maintenance and expansion medium of iPSCs for 16 to 20 days after electroporation.

In step I), the initial culture medium of iPSCs may be a medium containing DMEM/F-12, HEPES, nerve growth factor, cell growth factor, and vitamins, and is preferably N2B27 medium.

In step II), the maintenance and expansion medium of iPSCs may be a medium containing DMEM/F-12, nerve growth factor, L-glutamine, β-mercaptoethanol, and basic fibroblast growth factor, and is preferably E8F medium.

In the present invention, the piPSC cells or populations formed in step 3) can be cultured with feeder cells from day 21 to 30, preferably from day 21 to 28, more preferably from day 21 to 25, after electroporation.

In the present invention, in step 3), the feeder cells can be treated with mitomycin C (MMC).

Said MMC is treated to inhibit cell proliferation of feeder cells, inactivate feeder cells, and induce cell differentiation and specialization, and doxorubicin, hydroxyurea, campotothecin, 5-fluorouracil (5-FU), and mitoxantrone, which are suitable for this purpose, can also be treated.

The present invention provides porcine induced pluripotent stem cells prepared by the preparation method described above.

In the present invention, the porcine induced pluripotent stem cells may be derived from somatic cells of a pig, and may be derived from, for example, fibroblasts, keratinocytes, blood cells, myocytes, and hepatocytes. Preferably, the porcine induced pluripotent stem cells may be derived from fibroblasts and characterized by expressing one or more pluripotency marker genes selected from the group consisting of Oct4, Sox2, Klf4, and c-Myc.

In the present invention, the porcine induced pluripotent stem cells can be stably maintained for at least 60, 70, 80, 90, and 100 passages, and since the episomal vector is not integrated into the cells during the passages, hOCT3, hOCT4, hSK, hUL, mP53DD, and EBNA1 genes may not be expressed.

In the present invention, the porcine induced pluripotent stem cells can have alkaline phosphatase (AP) activity.

In the present invention, the porcine induced pluripotent stem cells can express one or more pluripotency marker genes selected from the group consisting of Oct4, Sox2, Klf4, and c-Myc.

The present invention provides a cell composition comprising at least one of embryoid bodies, myogenic progenitors, myoblasts, or myotube cells differentiated from the porcine induced pluripotent stem cells prepared by the preparation method described above.

In the present invention, the term “embryoid body” refers to a stem cell-derived spherical cell mass produced in a suspension culture state, and has the potential to differentiate into endoderm, mesoderm, and ectoderm, and is thus used as a precursor in most differentiation induction processes to obtain tissue-specific differentiated cells.

In the present invention, the embryoid body can form teratoma through spontaneous differentiation.

Teratoma is a type of tumor that can develop in embryonic cells or stem cells, and is characterized by the disorderly growth of various types of cells. Teratoma is mainly used to evaluate the differentiation capacity and pluripotency of induced pluripotent stem cells (iPSCs).

In the present invention, the embryoid body can differentiate into various cell lineages such as hepatocytes, vascular endothelial cells, neural cells, and muscle cells, and preferably, can differentiate into muscle cells.

In the present invention, the embryoid body can be differentiated into muscle cells and used for cultured meat production.

The term ‘cultured meat’ used in the present invention refers to edible meat obtained by collecting cells from a living animal and then proliferating them using cell engineering technology.

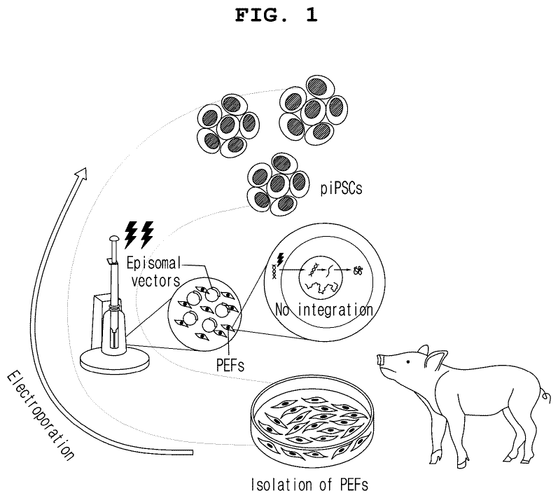

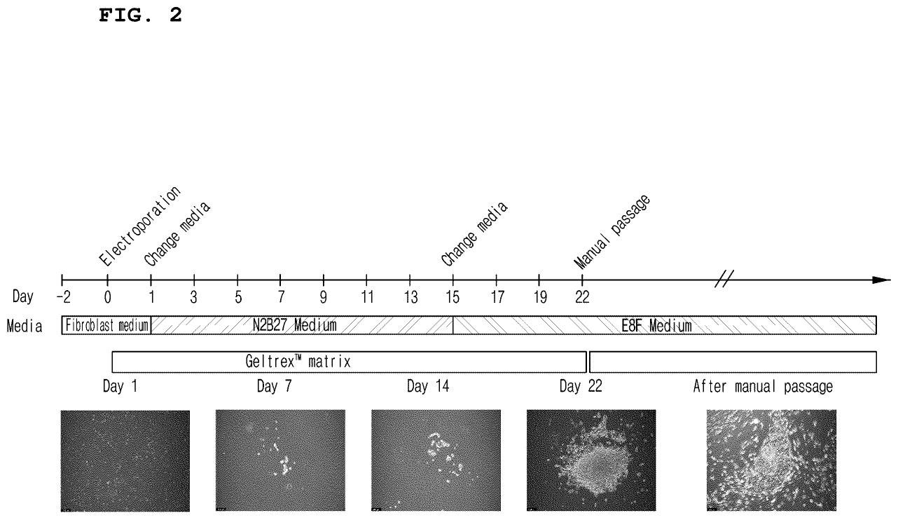

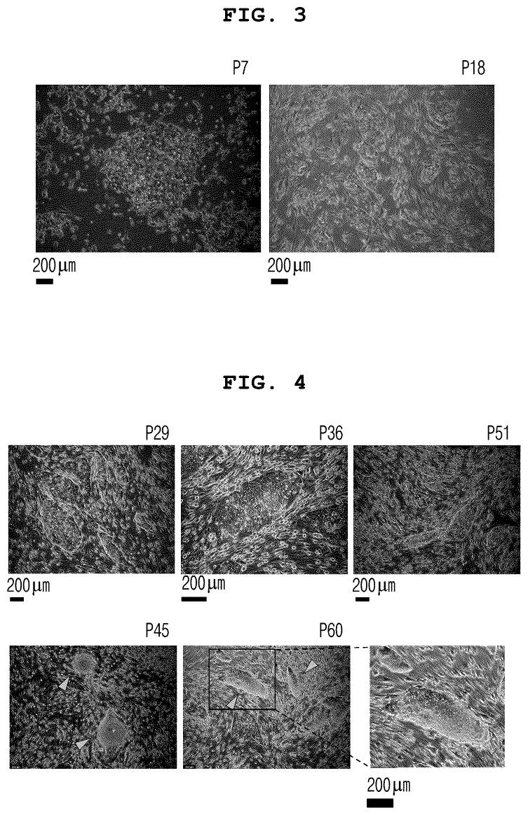

According to specific examples and experimental examples of the present invention, the present inventors used an approach in which foreign genes are not integrated throughout the entire process from piPSC generation to muscle differentiation. Specifically, reverse differentiated piPSCs were produced by introducing six types of episomal vectors (hOCT3, hOCT4, hSK, hUL, mP53DD, and EBNA1) into porcine embryonic fibroblasts (PEFs) by electroporation. After initial culture, piPSCs were stably maintained for a certain period of time through mechanical passage using culture media and feeder cells. Specifically, iPSCs were cultured in the initial culture medium for 1 to 15 days after electroporation, and then cultured in the maintenance and expansion medium for 16 to 20 days. On days 21 to 25 after electroporation, piPSCs were maintained by culturing with feeder cells. Maintenance of piPSCs was performed by transferring piPSCs to fresh STO feeder layers every 7 to 9 days using a mechanical passage method (see FIGS. 1 and 2). The piPSCs were separated by mechanical passaging after chemical separation up to passage 20, confirming that the mechanical method was effective in maintaining a more stable morphology during long-term culture (see FIGS. 3 and 4).

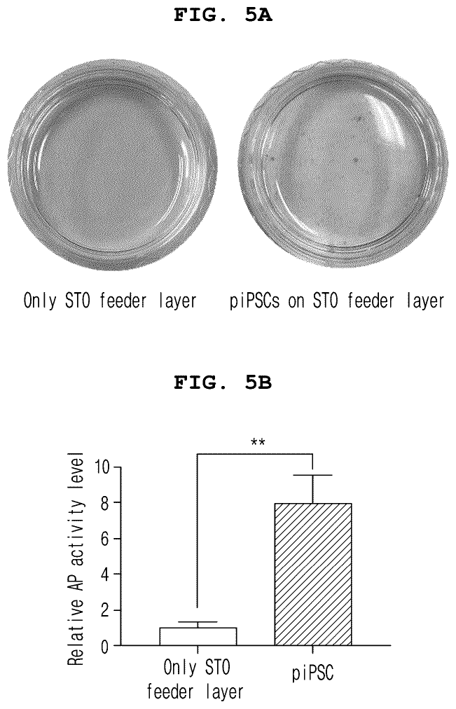

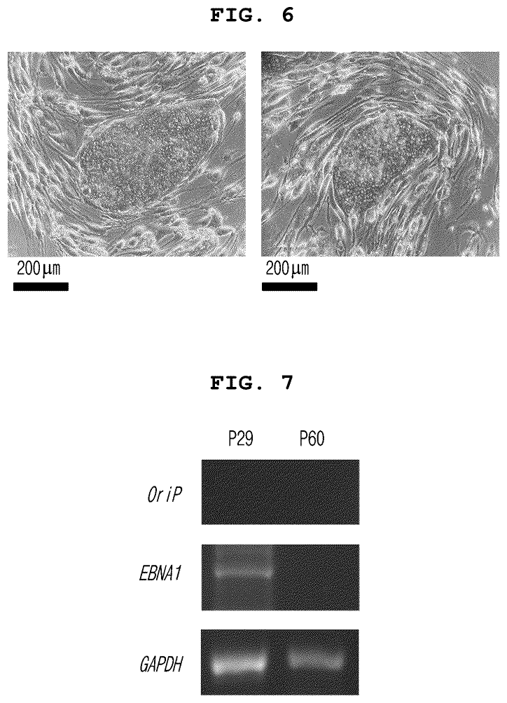

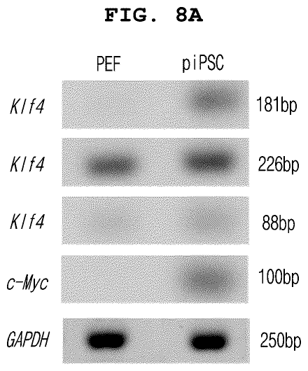

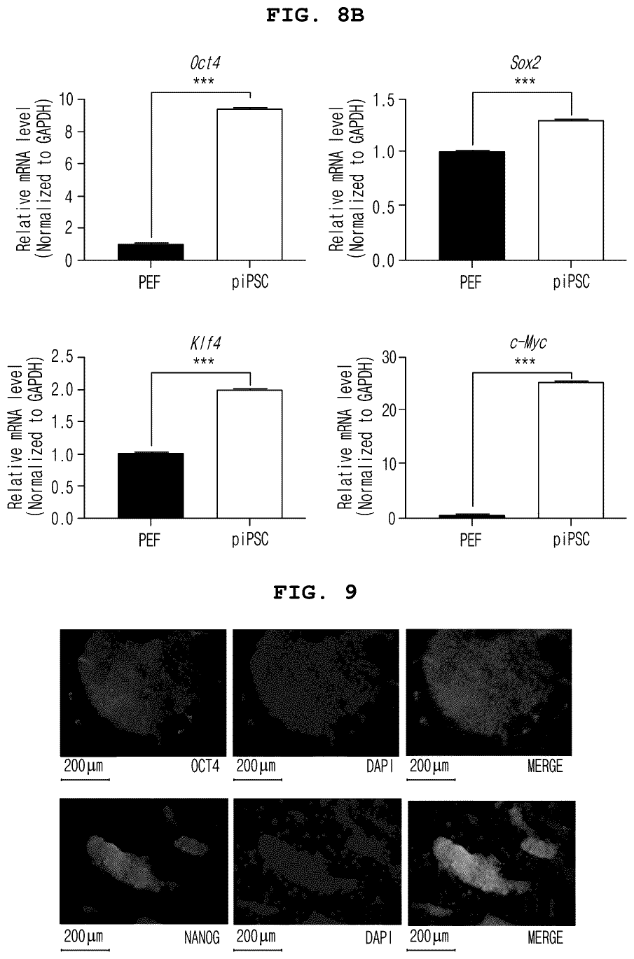

Integration of the episomal vector in piPSCs was confirmed by RT-PCR, and it was confirmed that the episomal vector was not integrated into the cells after multiple passages (see FIG. 7). In addition, the pluripotency of piPSCs was evaluated by AP staining (see FIGS. 5A, 5B and 6), and the pluripotency of piPSCs was confirmed through the expression levels of pluripotency marker genes Oct4, Sox2, Klf4, and c-Myc (see FIGS. 8A, 8B and 9).

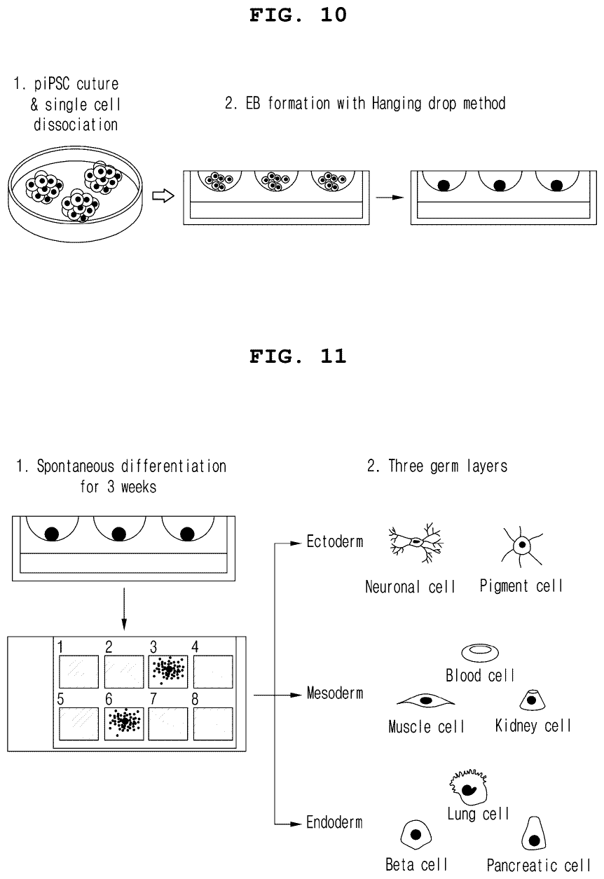

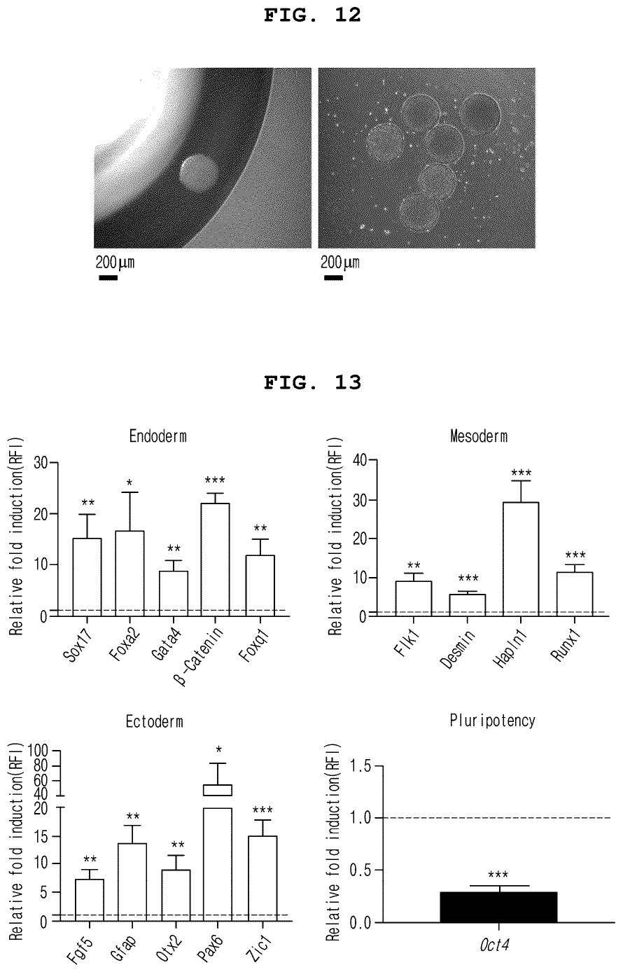

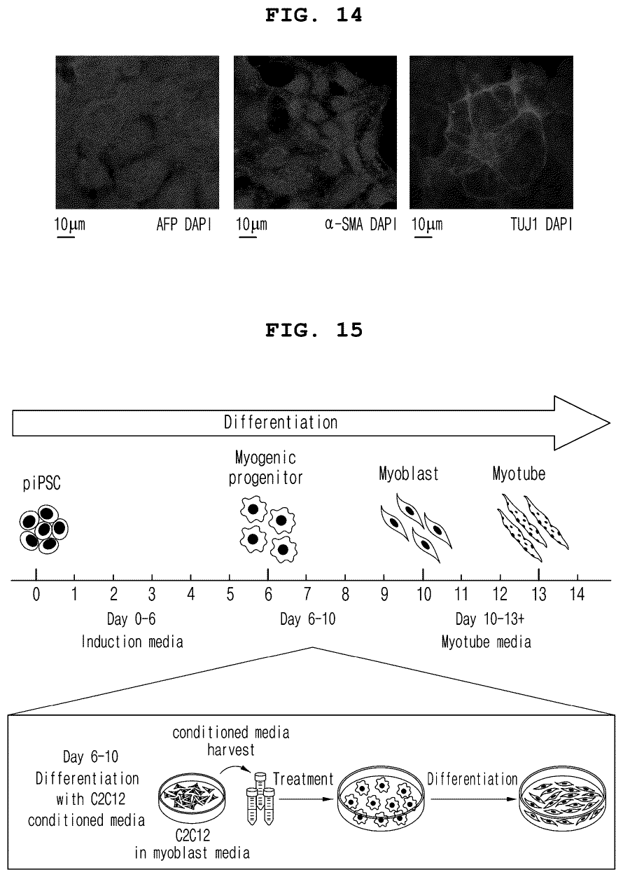

In addition, embryoid bodies (pEBs) were generated from piPSCs using the hanging drop method to confirm the spontaneous differentiation capacity of piPSCs (see FIG. 10), After 3 weeks of spontaneous differentiation, it was confirmed that pEBs can spontaneously differentiate into 3 different types of germ layers (ectoderm, mesoderm, and endoderm) (see FIGS. 11 and 12). Expression levels of endoderm differentiation markers (Sox17, Foxa2, Gata4, β-Catenin, and Foxq1), mesoderm differentiation markers (Flk1, Desmin, Hapln1, and Runx1), and ectoderm differentiation markers (Fgf5, Gfap, Otx2, Pax6, and Zic1) were compared in piPSCs and embryoid bodies. As a result, it was confirmed that the embryoid bodies generated from piPSCs can spontaneously differentiate into three germ layers. It was also confirmed that pluripotency was reduced by checking the expression of Oct4, an important transcription factor involved in maintaining pluripotency (see FIGS. 13 and 14).

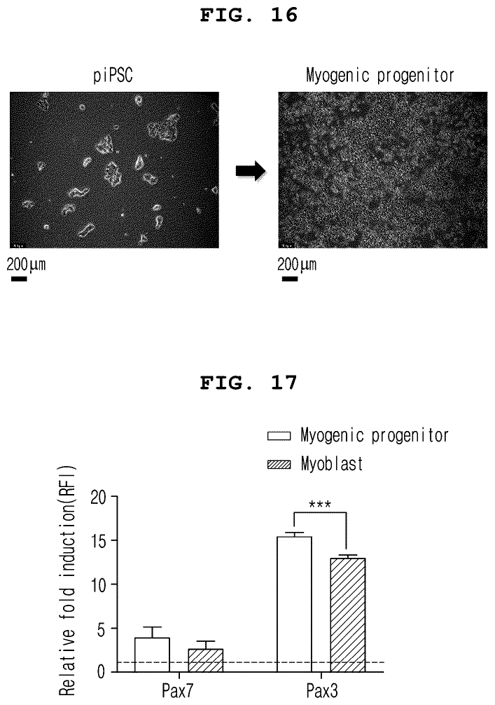

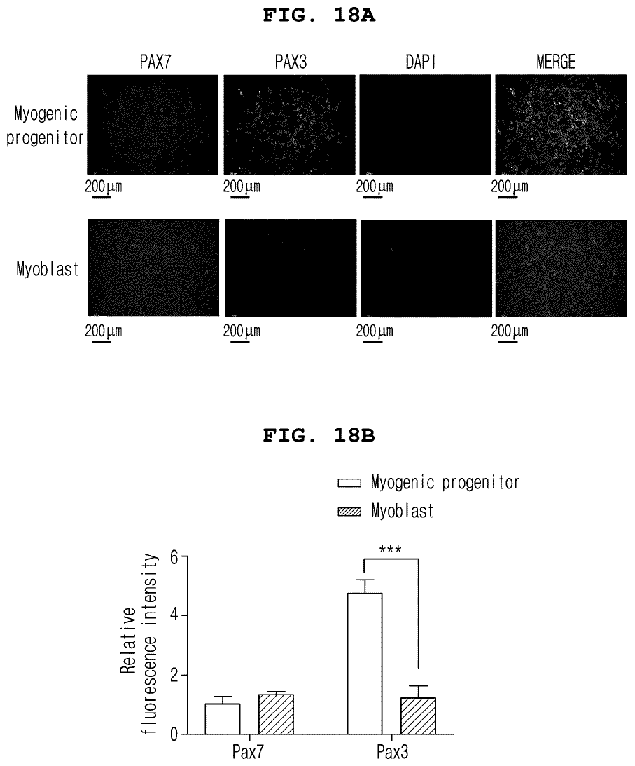

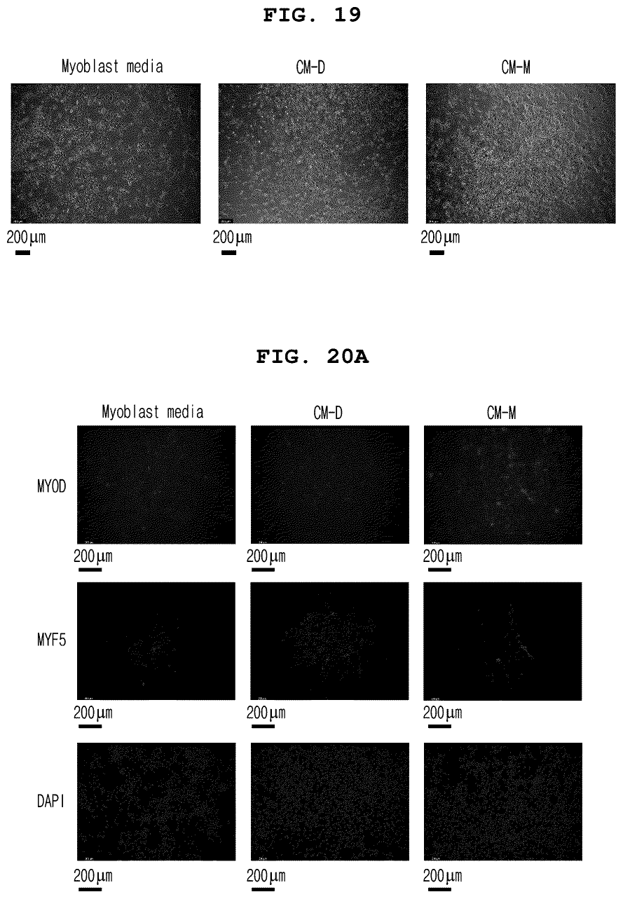

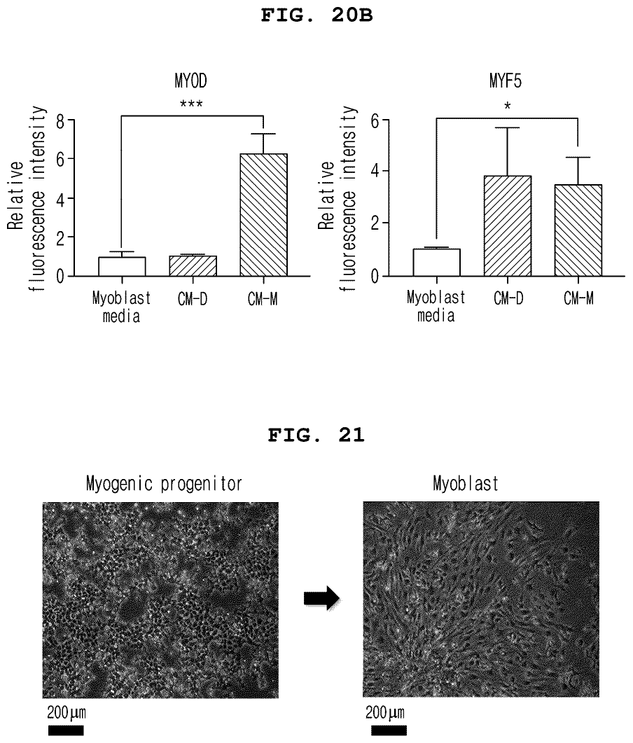

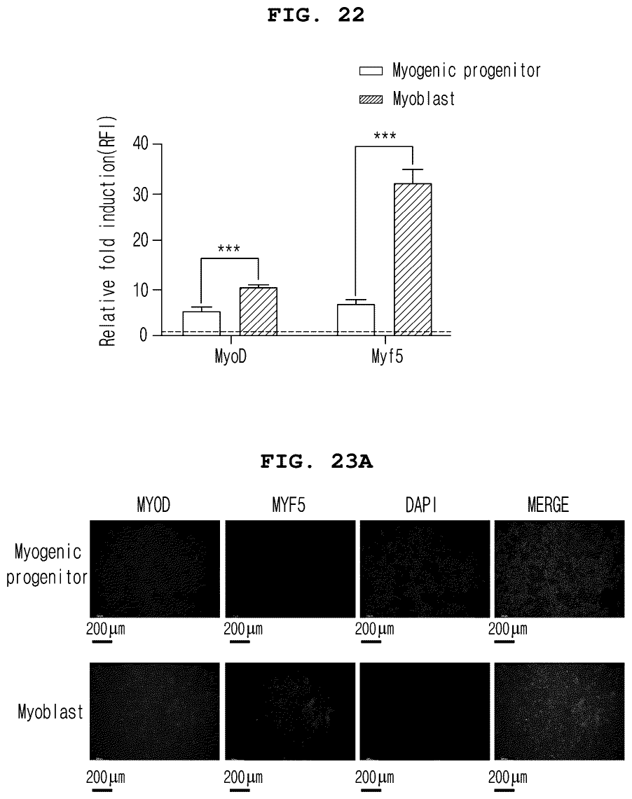

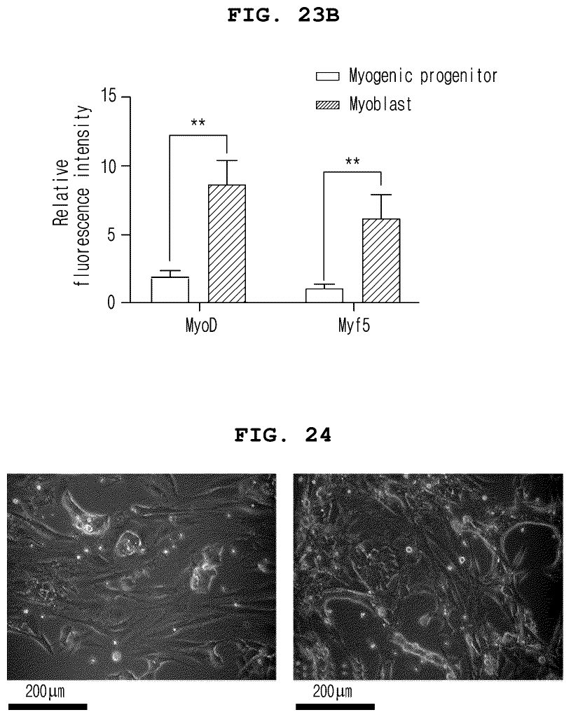

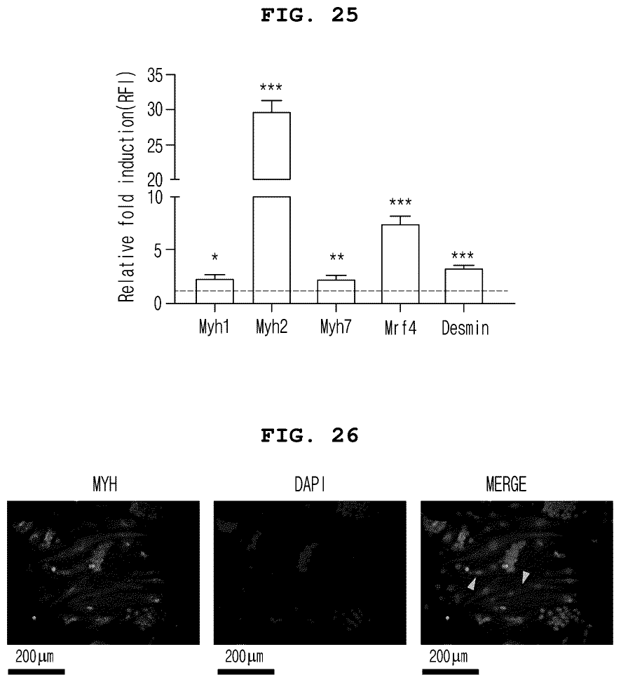

To differentiate piPSCs into myotube cells, iPSC differentiation medium and C2C12 conditioned medium (CM) derived from C2C12 cells were used (see FIGS. 15 and 16). To confirm the differentiation of piPSCs into myogenic progenitors, the expression of Pax3 and Pax7, which are highly expressed in the early stage of muscle differentiation, was investigated. As a result, it was confirmed that the differentiation into myogenic progenitors was induced (see FIGS. 17, 18A and 18B). To optimize myoblast differentiation, three culture medium conditions were tested, and the differentiation efficiency tended to be higher in the conditioned medium (CM-M) derived from C2C12 cells cultured in myoblast medium for 1 day (see FIGS. 19, 20A and 20B). Morphological changes from myogenic progenitors to myoblasts were confirmed (see FIG. 21). In addition, the degree of differentiation into myoblasts was confirmed through the expression of MyoD and Myf5, which are markers of myoblasts (see FIGS. 22, 23A and 23B). Finally, the differentiation into myotube cells was confirmed through microscopic images (see FIG. 24), and the final differentiation into myotube cells was confirmed through the expression of myotube cell markers Myh1, Myh2, Myh7, Mrf4, and Desmin (see FIGS. 25 and 26).

Hereinafter, the present invention will be described in detail by the following examples and experimental examples. However, the following examples and experimental examples are only intended to explain the present invention in more detail, and it is not intended that the scope of the present invention is limited and interpreted by the following description.

### <Preparative Example 1> Preparation of Porcine Induced Pluripotent Stem Cells (piPSCs)

### <1-1> Cell Culture Conditions

Porcine embryonic fibroblasts (PEFs) were provided by Seoul National University School of Dentistry. PEFs were cultured in Dulbecco's Modified Eagle Medium (DMEM, Cytiva, #SH30243.01) supplemented with 20% fetal bovine serum (FBS, Gibco, #26140-079) and 1% penicillin/streptomycin on 0.1% gelatin-coated culture dishes. STO feeder cells were prepared by inactivating with mitomycin C (Sigma Aldrich, #M0503) for 1 hour and 30 minutes, and seeded at 4˜5×10⁵ cells/well (35 pi dishes) on culture dishes coated with 0.1% gelatin. Porcine induced pluripotent stem cells (piPSCs) were cultured on STO feeder layer in Essential 8 Flex Medium Kit (E8F, Gibco, #A2858501) supplemented with 100 μg/ml of primocin (Invivogen, #ant-pm-05). The piPSCs were subcultured until P20 by passaging every 5-6 days using Versene solution (Gibco, #15040066). Thereafter, they were mechanically separated every 7-9 days. All cells were cultured in a 37° C., 5% CO₂ humidified environment.

<1-2> Preparation of piPSCs

PEFs were isolated using 0.25% Trypsin-EDTA (Gibco, #25200072) and then electroporated using the Neon™ Transfection System (Invitrogen). Cells were electroporated with the Epi5™ Episomal iPSC Reprogramming Kit (Invitrogen, #A15960) or the following five episomal vectors: 0.25 μg pCE-hOCT3/4 (Addgene, #41813), 0.25 μg pCE-hSK (Addgene, #41814), 0.25 μg pCE-hUL (Addgene, #41855), 0.33 μg pCE-mP53DD (Addgene, #41856), and 0.2 μg pCXB-EBNA1 (Addgene, #41857).

Specifically, PEFs were precipitated in EP tubes at a concentration of 2×10⁵ cells/well, resuspended in 10 μl of Buffer R (Invitrogen), and 1 μl of Tube A and Tube B of the Epi5™ Episomal iPSC Reprogramming Kit were added to each EP tube containing PEFs and resuspended. Cells and vectors in EP tubes were electroporated using the Neon™ Transfection system at 1650 V, 10 ms, and 3 pulses. The electroporated cells were cultured in culture dishes coated with 0.2% Geltrex (Gibco, #A1413302) containing a fibroblast medium (DMEM containing 20% FBS and 1% penicillin/streptomycin) for 1 day. From the next day to 15 days after electroporation, the medium was changed to N2B27 medium (DMEM/F-12, containing HEPES, Gibco, #11330032). Said N2B27 medium is supplemented with the following components: 1×N-2 Supplement (Gibco, #17502048), 1×B-27™ Supplement (Gibco, #17504044), 1×MEM non-essential amino acids (NEAA, Gibco, #11140050), 2 mM GlutaMAX™ Supplement (Gibco, #35050061), 0.1 mM 2-Mercaptoethanol (Gibco, #21985023), 1% Penicillin/Streptomycin, 100 ng/mL Basic fibroblast growth factor (bFGF).

On the 16th day after electroporation, the medium was changed to Essential 8™ Flex Medium Kit (E8F, Gibco) supplemented with 100 μg/ml Primocin, and on the 21st day, the cells were transferred to STO feeder layer using Versene solution. To maintain piPSC colonies, they were transferred to fresh STO feeder layer by mechanical passage once every 7 to 9 days (see FIGS. 1 and 2).

### <Experimental Example 1> Analysis of piPSC Characteristics

The piPSCs were separated every 5-6 days using a chemical separation method until P20, and then every 7-9 days using a mechanical passage method. However, when chemical separation methods were used, piPSCs showed instability, but maintained an aggregated morphology with clear boundaries after long-term culture (more than 60 days) (see FIG. 3). Through this, it was confirmed that piPSCs exhibited a more stable morphology when mechanical passage methods were used rather than chemical separation.

In addition, piPSCs exhibited a morphology similar to embryonic stem cells, characterized by rounded shape, large nuclei, and minimal cytoplasm (see FIG. 4). This shows that piPSCs can maintain their morphology during long-term culture, indicating that piPSCs have been successfully generated. During long-term culture, most piPSCs exhibited a flat, epithelial-like morphology (FIG. 4, top panel), but some piPSCs formed dome-shaped colonies (FIG. 4, bottom panel). The present inventors confirmed that piPSCs exhibit characteristics of both human ESCs and mouse ESCs.

### <Experimental Example 2> Evaluation of Pluripotency of piPSCs (AP Staining)

To evaluate the pluripotency of piPSCs, the alkaline phosphatase (AP) activity of piPSCs (P12, P29) was confirmed using AP staining Kit II (STEMGENT, #00-0055). PiPSCs were cultured on STO feeder cells for 7 to 9 days, and then the culture medium was removed. Afterwards, the cells were washed with 0.05% PBS-T (Triton X-100) and fixed with fixing solution for 2-5 minutes at room temperature. After removing the fixing solution, the cells were washed again with 0.05% PBS-T and stained with AP substrate solution for 15 minutes at room temperature in the dark. To stop the reaction, the AP substrate solution was removed and the cells were washed twice with PBS. Finally, the purple-stained cells were observed under a microscope.

Alkaline phosphatase (AP), which is abundant in pluripotent stem cells, is often used as a general marker of stem cells. AP staining results showed that there were more AP-positive cells in the piPSC group than in the STO feeder layer-only group, and AP expression in piPSCs was approximately 7.9 times higher than in the STO feeder layer-only group (see FIGS. 5A and 5B). When observed under a microscope, the piPSCs were identified as purple and AP-positive, while the surrounding STO feeder layer exhibited AP-negative characteristics (see FIG. 6). This suggests that the piPSCs exhibit the characteristic features of stem cells.

### <Experimental Example 3> Evaluation of Integration of Episomal Vectors in piPSCs

When piPSCs were generated by electroporation using episomal vectors, gene expression was analyzed by reverse transcription-PCR (RT-PCR) to confirm whether the episomal vectors were integrated into the cells.

First, total RNA was extracted from cells using TRIzol™ reagent (Invitrogen, #15596018). RNA concentration was measured using a NanoDrop™ One/OneC UV/Vis spectrophotometer (Thermo Scientific). Then, cDNA was synthesized using TOPscript™ RT DryMIX (Enzynomics, #RT200). The primers used are listed in Table 1.

TABLE 1

| Gene | Forward (5′-3′) | Reverse (5′-3′) |
|---|---|---|
| EBNA1 | ATC GTC AAA GCT GCA | CCC AGG AGT CCC AGT |
| | CAC AG | AGT CA |
| OriP | TTC CAC GAG GGT AGT | TCG GGG GTG TTA GAG |
| | GAA CC | ACA AC |

Reverse transcription PCR (RT-PCR) amplification for 35 cycles was performed using PCR Premix (TOYOBO, #KMM-201). Amplification conditions are as follows; predenaturation at 95° C. for 5 minutes, denaturation at 98° C. for 10 seconds, annealing at 54-60° C. for 30 seconds, elongation at 70° C. for 1 minute, and final extension at 70° C. for 5 minutes. Electrophoresis was performed on a 1.5% agarose gel supplemented with GelRed® (#41003), and images were acquired using a DavinchChemi™ Imager (Davinch-K). Quantitative reverse transcription PCR (qRT-PCR) amplification was performed using a 7500 Real-Time PCR System (Applied Biosystems). The PCR mixture contained 10 μl TOPreal™ SYBR Green qPCR PreMIX (Enzynomics, #RT500), 1 μl cDNA, 2 μl total primers, and 8 μl sterile water, and experiments were performed in triplicate. The mRNA levels were normalized to the housekeeping gene GAPDH, and the relative expression levels of the target genes were quantified using the 2^{-ΔΔCt} method.

Analysis of gene expression using reverse transcription PCR (RT-PCR) revealed that the OriP gene was not detected in the early-stage P29 piPSCs, but the EBNA1 gene was detected. However, neither gene was detected in the later P60 piPSCs (see FIG. 7). Thus, after multiple passages, it was confirmed that the episomal vectors were not integrated into the cells.

### <Experimental Example 4> Evaluation of Pluripotency of piPSCs (Confirmation of Gene Expression)

To confirm the pluripotency of piPSCs at the gene level, RT-PCR was performed using the same method as in Experimental Example 3. The primers used were porcine specific primers and are listed in Table 2.

TABLE 2

| Gene | Forward (5′-3′) | Reverse (5′-3′) |
|---|---|---|
| GAPDH | GTCGGAGTGAACGGATTTG | CACCCCATTTGATGTTGGC |
| | GC | G |
| Oct4 | CGAGGAGTCCCAGGACATC | ACTGAGCTGCAAAGCCTCA |
| | AA | A |
| Sox2 | ATGGGCTCAGTGGTCAAGT | AGAGAGGCAGTGTACCGTT |
| | C | G |
| Klf4 | GGTGCGGAGGAACTGCTAA | GGTAGTTTGGCCCATGGTG |
| | G | G |
| c-Myc | TCCACGAAACTTTGCCCAC | CAGAATAGCCTCTCCGCGT |
| | T | C |

As a result of confirming the expression of pluripotency genes Oct4, Sox2, Klf4, and c-Myc, the expression of Oct4 and c-Myc genes was significantly increased in piPSCs. Compared to PEFs, Oct4 was found to increase 9.3-fold and c-Myc was found to increase 25.9-fold. Sox2 and Klf4 genes were expressed at a certain level in PEFs, but were relatively more highly expressed in piPSCs, with Sox2 expression increasing 1.2-fold and Klf4 expression increasing 1.9-fold (see FIGS. 8A and 8B).

### <Experimental Example 5> Evaluation of Pluripotency of piPSCs at Protein Level

The pluripotency of piPSCs was evaluated at the protein level using immunocytochemistry (ICC).

First, cell samples were fixed in 4% paraformaldehyde (PFA) PBS solution at room temperature for 10 minutes, then the fixing solution was removed and washed three times with PBS. Afterwards, the fixed cells were permeabilized using 0.25% PBS-T for 10 minutes. The samples were washed three times with 0.01% PBS-T and blocked with a mixture of 3% BSA and 0.1% PBS-T at room temperature for 1 hour. After blocking, the samples were washed with PBS-T and cultured overnight at 4° C. on a rocker with the primary antibodies in a mixture of 1% BSA and 0.1% PBS-T. Anti-OCT4 antibody (1:500, Abcam, #ab18976) and anti-NANOG antibody (1:500, Abcam, #ab109250) were used as the primary antibodies. After culture with the primary antibodies, the cells were cultured with the secondary antibodies in a mixture of 1% BSA and 0.1% PBS-T in the dark for 1 hour at room temperature. As the secondary antibodies, Alexa 594 (1:1000, Abcam, #ab150080) or Alexa 488 (1:1000, Invitrogen, A-11008) conjugated antibodies were used. Afterwards, the cells were washed three times with 0.1% PBS-T and stained with 1 μg/ml of DAPI for 5 minutes to visualize the nuclei. Images were obtained using Leica DMi8 and STELLARIS 8 STED & FALCON confocal microscopes (Leica Microsystems).

As a result, the pluripotency was confirmed at the protein level by observing that Oct4 and Nanog, the key transcription factors that maintain pluripotency, were expressed in the nuclei (see FIG. 9).

### <Experimental Example 6> Confirmation of Spontaneous Differentiation Capacity of piPSCs

Embryoid bodies (pEBs) formed from piPSCs were generated using the traditional hanging drop method (see FIG. 10). The hanging drop method is a traditional technique in which cells are suspended upside down in a water droplet and gravity causes them to gather at the bottom of the droplet to form a spherical structure. First, piPSCs were cultured on STO feeder layers for 7-9 days. Afterwards, they were mechanically separated and re-seeded into culture dishes coated with Geltrex (Gibco, #A1413302). After culturing for 7-9 days, cells were dissociated using Accutase solution (Sigma-Aldrich, #A6964). Then, embryoid bodies (EBs) were formed by the hanging drop method in a medium containing DMEM/F12 (Kibo, #11320-033), 20% KnockOut Serum Replacement (Kibo, #10828028), 1 mM GlutaMax, 0.1 mM 2-mercaptoethanol, 1× NEAA, and 1% penicillin/streptomycin. Five days later, EBs were transferred to confocal dishes coated with 0.2% gelatin and allowed to undergo spontaneous differentiation for 3 weeks in DMEM supplemented with 10% FBS and 1% penicillin/streptomycin.

After three weeks of spontaneous differentiation, it was confirmed that pEBs could spontaneously differentiate into three germ layers (see FIG. 11).

In addition, as a result of EB (embryonic body) formation from piPSCs, pEBs were formed from piPSCs for 5 days in the absence of bFGF, and the pEBs exhibited a spherical shape with a uniform size and a size of 400 to 500 μm (see FIG. 12).

<6-1> Confirmation of Spontaneous Differentiation Capacity of piPSCs Through qRT-PCR

To evaluate the spontaneous differentiation capacity of piPSCs, the differentiation ability of piPSCs was evaluated by qRT-PCR. qRT-PCR was performed using the same method as in Experimental Example 3, and the primers used are listed in Table 3.

TABLE 3

| Gene | Forward (5′-3′) | reverse (5′-3′) |
|---|---|---|
| GAPDH | GTCGGAGTGAACGGATTT | CACCCCATTTGATGTTGGC |
| | GGC | G |
| Oct4 | CGAGGAGTCCCAGGACAT | ACTGAGCTGCAAAGCCTC |
| | CAA | AA |
| Sox 17 | CATGACTCCGGTGTGAAT | CAGTAATATACCGCGGAG |
| | CTC | CTG |
| Foxa2 | CACGTACATGAGCATGTC | ACTCAAGTGCGGCCCCAT |
| | GG | |
| Gata4 | AAAGAGGGGATCCAAACC | TTGCTGGAGTTGCTGGAA |
| | AG | G |
| β- | TGTAGAAACAGCTCGTTG | ACAGAATCCACTGGTGAA |
| Catenin | TACC | CC |
| Foxq1 | TGGCGGAGATCAACGAGT | TGTACTCGCTGTTGGGGTT |
| | A | |
| Flk1 | ACACTGGAGCCTACAAGT | CTCAGTAATGTACACGAC |
| | G | TCC |
| Desmin | CCTCAACTTCCGAGAAAC | TCACTGACGACCTCCCCA |
| | AAGC | TC |
| Hapln1 | ATCTCTAGGCCAAGAAGG | TGCTCTGAAGCAGTAGAC |
| | C | AC |
| Runx1 | CTCAGAGTCAGATGCAGG | TGAGAGTCGACTGGAAAG |
| | A | TTC |
| Fgf5 | TCTACTGCAGAGTGGGCA | ATGTATTGCTGAGGCATA |
| | T | GG |
| Gfap | GAGAACAACCTGGCTGCC | CTCATACTGCGTGCGGAT |
| | TA | CT |
| Otx2 | AACCGCCTTACGCAGTCA | CTTAAACCATACCTGCAC |
| | AT | CC |
| Pax6 | GCAGCCAAAATAGATCTA | GAGCGCTGTAGGTGTTTG |
| | CCTG | TG |
| Zic1 | AGCGACAAGCCCTATCTT | CGTGGACCTTCATGTGTTT |
| | TG | G |

Endoderm differentiation was confirmed using markers such as Sox17, Foxa2, Gata4, and βFoxq1, while mesoderm differentiation was evaluated using Flk1, Desmin, Hapln1, and Runx1. In addition, ectoderm differentiation was evaluated using Fgf5, Gfap, Otx2, Pax6, and Zic1, and the Oct4 gene was used to confirm the reduction of pluripotency.

As a result, gene expression of Gata4 and β-Catenin was increased 8.7-fold and 21.9-fold, respectively, in the endoderm compared to piPSCs, while gene expression of Desmin and Hapln1 was increased more than 5.8-fold and 29.5-fold, respectively, in the mesoderm compared to piPSCs. In the ectoderm, gene expression of Fgf5 and Pax6 was increased 7.3-fold and up to 54.8-fold, respectively, compared to piPSCs.

In addition, evaluation of pluripotency using Oct4, an important transcription factor related to the maintenance of pluripotent cells, revealed a decrease in pluripotency (see FIG. 13).

<6-2> Confirmation of Spontaneous Differentiation Capacity of piPSCs at Protein Level

The expression of proteins for the three germ layers was confirmed by immunocytochemistry (ICC) using the same method as in Experimental Example 5. Alpha-fetoprotein (AFP) for the endoderm, α-smooth muscle actin (α-SMA) for the mesoderm, and Class III beta-tubulin (TUJ1) for the ectoderm were used as the primary antibodies.

As a result, it was confirmed that all markers for the three germ layers were expressed after differentiation (see FIG. 14). Through this, it was confirmed that piPSCs had differentiation ability.

### <Example 1> Differentiation Process from piPSCs to Myotubes

During muscle differentiation, piPSCs go through three stages to finally form myotube cells. Differentiation was promoted using iPSC differentiation medium and conditioned medium (CM) derived from C2C12 cells. First, myogenic progenitor cells were formed through a six-day induction process, and then differentiated into myoblasts using CM for three to four days. Myoblasts were differentiated into myotube cells in myotube medium for 3-4 days (see FIG. 15).

Specifically, piPSCs were cultured on STO feeder layer for 7-9 days. Afterwards, they were mechanically separated and re-dispensed into culture dishes coated with Geltrex. After additional culturing for 7-9 days, cells were separated using Accutase solution. The separated cells were seeded at a density of 4×10⁴ cells/well in 6-well plates coated with Collagen type 1 (Sigma-Aldrich, #C7661). Collagen type 1 coating was performed for at least 4 hours before seeding the cells. Subsequently, differentiation into myogenic progenitors was induced for 6 days using induction medium (Amsbio, #SKM-01), and 2 ml of medium was used in each well. Myogenic progenitors were separated using 0.25% trypsin-EDTA and then replated at a density of 4×10⁴ cells/well in 12-well plates coated with collagen type 1. Afterwards, myogenic progenitors were cultured in myoblast media (Amsbio, #SKM-02) for 1 day and then differentiated into myoblasts in conditioned media from C2C12 cells cultured in myoblast media (CM-M) for 3-4 days. Then, the myoblasts were cultured in myotube medium (Amsbio, #SKM-03) for 3-4 days to further differentiate them into myotubes.

Under feeder-free conditions, piPSCs showed a round and dense morphology, but when induced into myogenic progenitors, they showed a relatively angular satellite cell-like morphology and appeared as individual cells rather than in a dense morphology (see FIG. 16).

### <Experimental Example 7> Differentiation of piPSCs into Myogenic Progenitors

<7-1> Confirmation of Differentiation of piPSCs into Myogenic Progenitors Through qRT-PCR

To confirm the differentiation of piPSCs into myogenic progenitors, the expression of Pax3 and Pax7 was confirmed by qRT-PCR. qRT-PCR was performed using the same method as in Experimental Example 3, and the primers used are listed in Table 4.

TABLE 4

| Gene | Forward (5′-3′) | reverse (5′-3′) |
|---|---|---|
| Pax3 | AAGCCCAAGCAGGTGACAA | CAGACCGCGTCCTTGAGTA |
| | C | G |
| Pax7 | CACTGTGCCCTCAGGTTTA | TCCTTCTTGTCCGCTTCGTC |
| | GT | |

Pax3 and Pax7 are known to be highly expressed in the early stages of muscle differentiation. The expression of Pax3 and Pax7 in myogenic progenitors was confirmed by qRT-PCR and was found to be 15.5- and 4.0-fold higher, respectively, compared to the expression in piPSCs. When comparing myogenic progenitors and myoblasts, there was no significant difference in Pax7 gene expression between myogenic progenitors and myoblasts, but Pax3 gene expression was significantly reduced as differentiation progressed (see FIG. 17).

<7-2> Confirmation of Differentiation of piPSCs into Myogenic Progenitors at Protein Level

To confirm the differentiation of piPSCs into myogenic progenitors at the protein level, immunocytochemistry (ICC) was performed using the same method as in Experimental Example 5. At this time, the myogenic progenitor markers PAX3 and PAX7 were used as primary antibodies.

As a result, there was no significant change in PAX7 expression, but PAX3 expression was significantly decreased as the cells progressed to the muscle cell stage (see FIGS. 18A and 18B).

### <Experimental Example 8> Differentiation of Myogenic Progenitors into Myoblasts

Several approaches have been used to optimize myoblast differentiation. The conditions used were as follows: myoblast media for iPSCs, conditioned media derived from C2C12 cells cultured in DMEM (DMEM conditioned media from C2C12 cells, CM-D), and conditioned media derived from C2C12 cells cultured in myoblast media for 1 day (CM-M).

The cells cultured in myoblast media exhibited a morphology similar to that of myogenic progenitors, while the cells cultured in CM-D appeared more pointed, and the cells cultured in CM-M tended to be much longer (see FIG. 19).

<8-1> Confirmation of Differentiation Efficiency from Myogenic Progenitors to Myoblasts at Protein Level

To confirm the differentiation efficiency from myogenic progenitors to myoblasts at the protein level, immunocytochemistry (ICC) was performed using the same method as in Experimental Example 5. At this time, MYOD and MYF5 were used as primary antibodies.

When the efficiency of muscle cell differentiation was evaluated by immunocytochemistry (ICC) under each medium condition, the MYOD protein expression level was similar in myoblast medium and CM-D, but in CM-M, the MYOD protein expression was approximately 6.3 times higher than in myoblast medium. MYF5 showed the lowest expression in myoblast medium, similar levels in CM-D and CM-M, and approximately 3.5 times higher expression in CM-M than in myoblast medium (see FIGS. 20A and 20B).

### <Experimental Example 9> Comparison of Differentiation Between Myogenic Progenitors and Myoblasts

Myoblast differentiation was performed under CM-M condition. As a result of confirming the morphological change from myogenic progenitors to myoblasts, myoblasts exhibited a more elongated shape compared to myogenic progenitors.

However, under the same conditions, it was confirmed that not all cells differentiated and some remained as myogenic progenitors (see FIG. 21).

<9-1> Confirmation of Myoblast Marker Expression Through qRT-PCR

To determine the degree of differentiation from myogenic progenitors into myoblasts through the expression of myoblast markers, the expression of MyoD and Myf5 was confirmed by qRT-PCR. qRT-PCR was performed using the same method as in Experimental Example 3, and the primers used are listed in Table 5.

TABLE 5

| Gene | Forward (5′-3′) | reverse (5′-3′) |
|---|---|---|
| MyoD | AAACGCAAGACCACTAACG | GAAGGCCTCGTTGACTTTG |
| | C | C |
| Myf5 | GACGAGTTTGAGCCACGAG | GTGGATTTCCTCTTGCACG |
| | T | C |

MyoD and Myf5 have been reported as typical markers of myoblasts. Through microscopic observation, it was confirmed that although not all cells were differentiated, the expression of MyoD and Myf5 in myoblasts was increased at the gene level compared to myogenic progenitors. In addition, compared to piPSCs, MyoD expression in myoblasts was increased 10.3-fold and Myf5 expression was increased 32.0-fold (see FIG. 22).

### <9-2> Confirmation of Myoblast Marker Expression at Protein Level

To confirm the expression of myoblast markers at the protein level, immunocytochemistry (ICC) was performed using the same method as in Experimental Example 5. At this time, MYOD and MYF5 were used as primary antibodies.

The expression of MYOD and MYF5 in myogenic progenitors and myoblasts was evaluated by immunocytochemistry (ICC), and as a result, MYOD and MYF5 were shown to be expressed more highly in myoblasts than in myogenic progenitors at the protein level (see FIGS. 23A and 23B).

### <Experimental Example 10> Final Differentiation into Myotubes

Myotube differentiation, the final stage of muscle differentiation, was confirmed through microscopic imaging. Muscle cells exhibited a long, tubular shape like muscle fibers, and had a characteristic morphology with two or more nuclei (see FIG. 24).

<10-1> Confirmation of Final Differentiation into Myotubes Through qRT-PCR

To confirm the final differentiation into myotubes, the expression of myotube markers Myh1, Myh2, Myh7, Mrf4, and Desmin was confirmed by qRT-PCR. qRT-PCR was performed using the same method as in Experimental Example 3, and the primers used are listed in Table 6.

TABLE 6

| Gene | Forward (5′-3′) | reverse (5′-3′) |
|---|---|---|
| Desmin | CCTCAACTTCCGAGAAACA | TCACTGACGACCTCCCCAT |
| | AGC | C |
| Myh1 | GGCAAGCAAGCATTCACA | GGCACTCTTGGCCTTGATC |
| | CA | T |
| Myh2 | GGACCCCCTGAATGACAC | CGGTCTGGAAGGAAGAAC |
| | AG | CC |
| Myh7 | ACCAACCTGTCCAAGTTCC | AGGACTGGGAGCTTTGTTG |
| | G | C |
| Mrf4 | CAGCCTTGGGCTTTTCTTC | AGGGCACAAAAGGATCAC |
| | G | CA |

As a result, the expression of myotube markers was significantly increased overall compared to that in myoblasts. Specifically, at the gene level, Myh1 was increased 1.8-fold, Myh7 was increased 2.1-fold, Mrf4 was increased 7.4-fold, Desmin was increased 3.3-fold, and Myh2 was increased the most by 29.7-fold compared to those in myoblasts (see FIG. 25).

<10-2> Confirmation of Final Differentiation into Myotubes at Protein Level

To confirm the terminal differentiation into myotubes at the protein level, immunocytochemistry (ICC) was performed using the same method as in Experimental Example 5. At this time, MYH was used as a primary antibody.

MYH protein is abundant in adult muscle fibers and plays an important role in determining the contraction speed of muscle fibers.

The expression of MYH was also confirmed at the protein level by immunocytochemistry (ICC), and myotubes with a fiber-like staining pattern and two or more nuclei were identified (see FIG. 26).

1. A method for preparing porcine induced pluripotent stem cells (piPSCs) comprising the following steps:
i) a step of transforming porcine embryonic fibroblasts (PEFs) with an episomal vector by electroporation to obtain transformed cells, wherein, the episomal vector is a vector expressing one or more genes selected from the group consisting of hOCT3/4, hSK, hUL, mP53DD, and EBNA1 genes; and
ii) a step of reverse differentiating the obtained transformed cells and then forming piPSC cells or populations.

2. The method for preparing porcine induced pluripotent stem cells (piPSCs) according to claim 1, wherein the method further comprises, after step 2, a step (step 3) of culturing the formed piPSC cells or populations with feeder cells to expand and maintain the piPSC cells or populations.

3. The method for preparing porcine induced pluripotent stem cells (piPSCs) according to claim 2, wherein the formed piPSC cells or populations are expanded and maintained by mechanical passage culture.

4. The method for preparing porcine induced pluripotent stem cells (piPSCs) according to claim 1, wherein the electroporation of step 1) is performed under the conditions of 1650 V, 10 ms, and 3 pulses.

5. The method for preparing porcine induced pluripotent stem cells (piPSCs) according to claim 1, wherein the transformed cells in step 2) is reverse-differentiated through a step of reverse differentiation comprising: I) a step of culturing in an initial culture medium of iPSCs for 1 to 15 days after electroporation; and II) a step of culturing in a maintenance and expansion medium of iPSCs for 16 to 20 days after electroporation.

6. The method for preparing porcine induced pluripotent stem cells (piPSCs) according to claim 5, wherein the initial culture medium of iPSCs of step I) is a medium comprising DMEM/F-12, HEPES, nerve growth factor, cell growth factor, and vitamins.

7. The method for preparing porcine induced pluripotent stem cells (piPSCs) according to claim 5, wherein the maintenance and expansion medium of iPSCs of step II) is a medium comprising DMEM/F-12, nerve growth factor, L-glutamine, β-mercaptoethanol, and basic fibroblast growth factor.

8. The method for preparing porcine induced pluripotent stem cells (piPSCs) according to claim 2, wherein the piPSC cells or populations formed in step 3) is cultured with feeder cells from day 21 to 25 after electroporation.

9. The method for preparing porcine induced pluripotent stem cells (piPSCs) according to claim 2, wherein the feeder cells are treated with mitomycin C (MMC).

10. The method for preparing porcine induced pluripotent stem cells (piPSCs) according to claim 3, wherein the mechanical passaging is a physical separation of cells or populations using a scraper or pipette.

11. The method for preparing porcine induced pluripotent stem cells (piPSCs) according to claim 3, wherein the mechanical passaging is performed every 5 to 10 days.

12. A porcine induced pluripotent stem cell prepared by the preparation method according to claim 1.

13. The porcine induced pluripotent stem cell according to claim 12, wherein the porcine induced pluripotent stem cell can be maintained for at least 100 passages.

14. The porcine induced pluripotent stem cell according to claim 12, wherein the porcine induced pluripotent stem cell does not have an episomal vector integrated into the cell.

15. The porcine induced pluripotent stem cell according to claim 14, wherein the porcine induced pluripotent stem cell does not express hOCT3/4, hSK, hUL, mP53DD, and EBNA1 genes.

16. The porcine induced pluripotent stem cell according to claim 12, wherein the porcine induced pluripotent stem cell has AP (alkaline phosphatase) activity.

17. The porcine induced pluripotent stem cell according to claim 12, wherein the porcine induced pluripotent stem cell expresses one or more pluripotency marker genes selected from the group consisting of Oct4, Sox2, Klf4, and c-Myc.

18. A cell composition comprising at least one of embryoid bodies, myogenic progenitors, myoblasts, or myotube cells differentiated from the porcine induced pluripotent stem cell obtained by the preparation method according to claim 1.

19. The cell composition according to claim 18, wherein the embryoid body forms teratoma through spontaneous differentiation.

20. The cell composition according to claim 18, wherein the cell composition differentiates into muscle cells.


SEQUENCE LISTING (58 sequences)

SEQ ID NO: 1 - DNA, 20 bp, synthetic construct
  source 1..20: mol_type = other DNA
atcgtcaaagctgcacacag

SEQ ID NO: 2 - DNA, 20 bp, synthetic construct
  source 1..20: mol_type = other DNA
cccaggagtcccagtagtca

SEQ ID NO: 3 - DNA, 20 bp, synthetic construct
  source 1..20: mol_type = other DNA
ttccacgagggtagtgaacc

SEQ ID NO: 4 - DNA, 20 bp, synthetic construct
  source 1..20: mol_type = other DNA
tcgggggtgttagagacaac

SEQ ID NO: 5 - DNA, 21 bp, synthetic construct
  source 1..21: mol_type = other DNA
gtcggagtgaacggatttggc

SEQ ID NO: 6 - DNA, 20 bp, synthetic construct
  source 1..20: mol_type = other DNA
caccccatttgatgttggcg

SEQ ID NO: 7 - DNA, 21 bp, synthetic construct
  source 1..21: mol_type = other DNA
cgaggagtcccaggacatcaa

SEQ ID NO: 8 - DNA, 20 bp, synthetic construct
  source 1..20: mol_type = other DNA
actgagctgcaaagcctcaa

SEQ ID NO: 9 - DNA, 20 bp, synthetic construct
  source 1..20: mol_type = other DNA
atgggctcagtggtcaagtc

SEQ ID NO: 10 - DNA, 20 bp, synthetic construct
  source 1..20: mol_type = other DNA
agagaggcagtgtaccgttg

SEQ ID NO: 11 - DNA, 20 bp, synthetic construct
  source 1..20: mol_type = other DNA
ggtgcggaggaactgctaag

SEQ ID NO: 12 - DNA, 20 bp, synthetic construct
  source 1..20: mol_type = other DNA
ggtagtttggcccatggtgg

SEQ ID NO: 13 - DNA, 20 bp, synthetic construct
  source 1..20: mol_type = other DNA
tccacgaaactttgcccact

SEQ ID NO: 14 - DNA, 20 bp, synthetic construct
  source 1..20: mol_type = other DNA
cagaatagcctctccgcgtc

SEQ ID NO: 15 - DNA, 21 bp, synthetic construct
  source 1..21: mol_type = other DNA
catgactccggtgtgaatctc

SEQ ID NO: 16 - DNA, 21 bp, synthetic construct
  source 1..21: mol_type = other DNA
cagtaatataccgcggagctg

SEQ ID NO: 17 - DNA, 20 bp, synthetic construct
  source 1..20: mol_type = other DNA
cacgtacatgagcatgtcgg

SEQ ID NO: 18 - DNA, 18 bp, synthetic construct
  source 1..18: mol_type = other DNA
actcaagtgcggccccat

SEQ ID NO: 19 - DNA, 20 bp, synthetic construct
  source 1..20: mol_type = other DNA
aaagaggggatccaaaccag

SEQ ID NO: 20 - DNA, 19 bp, synthetic construct
  source 1..19: mol_type = other DNA
ttgctggagttgctggaag

SEQ ID NO: 21 - DNA, 22 bp, synthetic construct
  source 1..22: mol_type = other DNA
tgtagaaacagctcgttgtacc

SEQ ID NO: 22 - DNA, 20 bp, synthetic construct
  source 1..20: mol_type = other DNA
acagaatccactggtgaacc

SEQ ID NO: 23 - DNA, 19 bp, synthetic construct
  source 1..19: mol_type = other DNA
tggcggagatcaacgagta

SEQ ID NO: 24 - DNA, 19 bp, synthetic construct
  source 1..19: mol_type = other DNA
tgtactcgctgttggggtt

SEQ ID NO: 25 - DNA, 19 bp, synthetic construct
  source 1..19: mol_type = other DNA
acactggagcctacaagtg

SEQ ID NO: 26 - DNA, 21 bp, synthetic construct
  source 1..21: mol_type = other DNA
ctcagtaatgtacacgactcc

SEQ ID NO: 27 - DNA, 22 bp, synthetic construct
  source 1..22: mol_type = other DNA
cctcaacttccgagaaacaagc

SEQ ID NO: 28 - DNA, 20 bp, synthetic construct
  source 1..20: mol_type = other DNA
tcactgacgacctccccatc

SEQ ID NO: 29 - DNA, 19 bp, synthetic construct
  source 1..19: mol_type = other DNA
atctctaggccaagaaggc

SEQ ID NO: 30 - DNA, 20 bp, synthetic construct
  source 1..20: mol_type = other DNA
tgctctgaagcagtagacac

SEQ ID NO: 31 - DNA, 19 bp, synthetic construct
  source 1..19: mol_type = other DNA
ctcagagtcagatgcagga

SEQ ID NO: 32 - DNA, 21 bp, synthetic construct
  source 1..21: mol_type = other DNA
tgagagtcgactggaaagttc

SEQ ID NO: 33 - DNA, 19 bp, synthetic construct
  source 1..19: mol_type = other DNA
tctactgcagagtgggcat

SEQ ID NO: 34 - DNA, 20 bp, synthetic construct
  source 1..20: mol_type = other DNA
atgtattgctgaggcatagg

SEQ ID NO: 35 - DNA, 20 bp, synthetic construct
  source 1..20: mol_type = other DNA
gagaacaacctggctgccta

SEQ ID NO: 36 - DNA, 20 bp, synthetic construct
  source 1..20: mol_type = other DNA
ctcatactgcgtgcggatct

SEQ ID NO: 37 - DNA, 20 bp, synthetic construct
  source 1..20: mol_type = other DNA
aaccgccttacgcagtcaat

SEQ ID NO: 38 - DNA, 20 bp, synthetic construct
  source 1..20: mol_type = other DNA
cttaaaccatacctgcaccc

SEQ ID NO: 39 - DNA, 22 bp, synthetic construct
  source 1..22: mol_type = other DNA
gcagccaaaatagatctacctg

SEQ ID NO: 40 - DNA, 20 bp, synthetic construct
  source 1..20: mol_type = other DNA
gagcgctgtaggtgtttgtg

SEQ ID NO: 41 - DNA, 20 bp, synthetic construct
  source 1..20: mol_type = other DNA
agcgacaagccctatctttg

SEQ ID NO: 42 - DNA, 20 bp, synthetic construct
  source 1..20: mol_type = other DNA
cgtggaccttcatgtgtttg

SEQ ID NO: 43 - DNA, 20 bp, synthetic construct
  source 1..20: mol_type = other DNA
aagcccaagcaggtgacaac

SEQ ID NO: 44 - DNA, 20 bp, synthetic construct
  source 1..20: mol_type = other DNA
cagaccgcgtccttgagtag

SEQ ID NO: 45 - DNA, 21 bp, synthetic construct
  source 1..21: mol_type = other DNA
cactgtgccctcaggtttagt

SEQ ID NO: 46 - DNA, 20 bp, synthetic construct
  source 1..20: mol_type = other DNA
tccttcttgtccgcttcgtc

SEQ ID NO: 47 - DNA, 20 bp, synthetic construct
  source 1..20: mol_type = other DNA
aaacgcaagaccactaacgc

SEQ ID NO: 48 - DNA, 20 bp, synthetic construct
  source 1..20: mol_type = other DNA
gaaggcctcgttgactttgc

SEQ ID NO: 49 - DNA, 20 bp, synthetic construct
  source 1..20: mol_type = other DNA
gacgagtttgagccacgagt

SEQ ID NO: 50 - DNA, 20 bp, synthetic construct
  source 1..20: mol_type = other DNA
gtggatttcctcttgcacgc

SEQ ID NO: 51 - DNA, 20 bp, synthetic construct
  source 1..20: mol_type = other DNA
ggcaagcaagcattcacaca

SEQ ID NO: 52 - DNA, 20 bp, synthetic construct
  source 1..20: mol_type = other DNA
ggcactcttggccttgatct

SEQ ID NO: 53 - DNA, 20 bp, synthetic construct
  source 1..20: mol_type = other DNA
ggaccccctgaatgacacag

SEQ ID NO: 54 - DNA, 20 bp, synthetic construct
  source 1..20: mol_type = other DNA
cggtctggaaggaagaaccc

SEQ ID NO: 55 - DNA, 20 bp, synthetic construct
  source 1..20: mol_type = other DNA
accaacctgtccaagttccg

SEQ ID NO: 56 - DNA, 20 bp, synthetic construct
  source 1..20: mol_type = other DNA
aggactgggagctttgttgc

SEQ ID NO: 57 - DNA, 20 bp, synthetic construct
  source 1..20: mol_type = other DNA
cagccttgggcttttcttcg

SEQ ID NO: 58 - DNA, 20 bp, synthetic construct
  source 1..20: mol_type = other DNA
agggcacaaaaggatcacca